(19) United States
(12) Patent Application Publication
(10) Pub. No.: US 2026/0345260 A1
(43) Pub. Date: Nov. 5, 2026

(54) SYSTEMS AND METHODS FOR ROBOTICALLY-ASSISTED HEMODIALYSIS ACCESS

(71) Applicant: Covidien LP, Mansfield, MA (US)

(72) Inventors: Max L. Balter, Boston, MA (US); Muhammad A. Ali, Morris Plains, NJ (US); Wenhao Yang, Sharon, MA (US); Justin K. Mann, Lake Elsinore, CA (US); Jeffrey H. Vogel, Brooklyn Park, MN (US); Jing Zhao, Superior, CO (US); Kaustubh R. Patil, Plymouth, MN (US)

(21) Appl. No.: 19/558,659

(22) Filed: Mar. 6, 2026

Related U.S. Application Data
(60) Provisional application No. 63/769,666, filed on Mar. 10, 2025.

(51) Int. Cl. A61B 34/30; A61B 17/34; A61B 34/20

(52) U.S. Cl. CPC A61B 34/30; A61B 17/3403; A61B 2017/3413; A61B 2034/2063

(57) ABSTRACT

A robotically-assisted hemodialysis access system includes a needle manipulation assembly having a frame, and a needle control assembly. The needle control assembly includes a needle carriage slidably connected to the frame, wherein a path of translation of the needle carriage defines an axis of translation; a needle drive motor operatively connected to the needle carriage, wherein a position of the needle carriage is controlled by the needle drive motor; and a pitch control mechanism including a pitch motor connected to operatively connected to the needle carriage, wherein an angle of inclination of the axis of translation of the needle carriage is controlled by the pitch motor. The needle control assembly also includes an imaging device.

### CROSS-REFERENCE TO RELATED APPLICATIONS

This application claims the benefit of the filing date of provisional U.S. Patent Application No. 63/769,666 filed on Mar. 10, 2025. The entire contents of the foregoing application are incorporated by reference herein.

### BACKGROUND

Vascular access is a critical first step in a range of diagnostic and therapeutic procedures, including drawing blood, administering fluids, introducing endovascular devices, and performing hemodialysis. In the U.S. alone, it is estimated that there are over 500,000 patients on hemodialysis, including in-clinic and at-home hemodialysis. This procedure requires two large bore needles to be placed into the vasculature of a patient so that blood can be withdrawn, filtered, and returned. While cannulation of the fistula and placement of the needles is generally a simple task, a significant number of cannulation complications occur, that not only impact a patient's quality of life, but are also a significant cost to the healthcare system. Technicians that work in dialysis centers are often undertrained, and historically, have had high turnover, which exacerbates the issue of cannulation. Likewise, it can be challenging for patients to teach themselves how to self-cannulate for home hemodialysis.

Accordingly, a need exists for an improved system for hemodialysis access, and methods for operating the same.

### SUMMARY

The present disclosure provides for a robotically-assisted hemodialysis access system including a controller; a display connected to the controller; and a needle manipulation assembly connected to the controller. The needle manipulation assembly includes a frame and a needle control assembly.

The needle control assembly includes a needle carriage slidably connected to the frame, wherein a path of translation of the needle carriage defines an axis of translation; a needle drive motor operatively connected to a first end of a cable, wherein a second end of the cable is operatively connected to the needle carriage, and wherein a position of the needle carriage is controlled by the needle drive motor; and a pitch control mechanism including a pitch motor operatively connected to the needle carriage, wherein an angle of inclination of the axis of translation of the needle carriage is controlled by the pitch motor.

The needle control assembly also includes an imaging device supported on a bracket, wherein the imaging device includes an imaging head defining a line of sight that crosses the axis of translation of the needle carriage.

### BRIEF DESCRIPTION OF THE DRAWINGS

Various embodiments of the present disclosure are described herein with reference to the drawings wherein:

FIG. 1 is a schematic illustration of a robotically-assisted hemodialysis access system according to an embodiment of the present disclosure;

FIG. 2 is a schematic illustration of a needle manipulation assembly of the system of FIG. 1, with a needle holder assembly posed at a first pitch orientation;

FIG. 2A is an enlarged view of the indicated area of detail of FIG. 2;

FIG. 3 is an enlarged view of the indicated area of detail of FIG. 2, with the needle holder assembly posed at a second pitch orientation;

FIG. 4 is a top, schematic view of the needle holder assembly posed at a first yaw angle;

FIG. 5 is a top, schematic view of the needle holder assembly posed at a second yaw angle;

FIG. 6 is a flow chart illustrating a work flow for using the robotically-assisted hemodialysis access system; and

FIG. 7 is an image of graphic user interface of the robotically-assisted hemodialysis access system.

### DETAILED DESCRIPTION

Embodiments of the presently disclosed robotically-assisted hemodialysis access system are described in detail with reference to the drawings, in which like reference numerals designate identical or corresponding elements in each of the several views. As used herein the term “distal” refers to the portion of the robotically-assisted hemodialysis access system and/or the needle coupled thereto that is closer to a patient, while the term “proximal” refers to the portion that is farther from the patient.

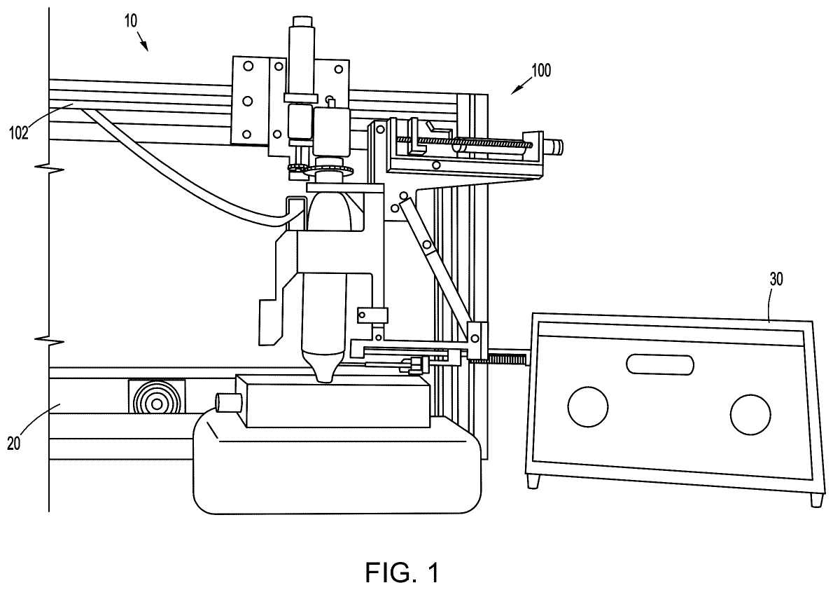

With reference to FIG. 1, a robotically-assisted hemodialysis access system, in accordance with the present disclosure, is generally designated as 10. The system 10 includes a controller or control processor 20, a display 30, and a needle manipulation assembly 100. Each of the display 30 and the needle manipulation assembly 100 are connected to the controller 20, either via wired or wireless connections.

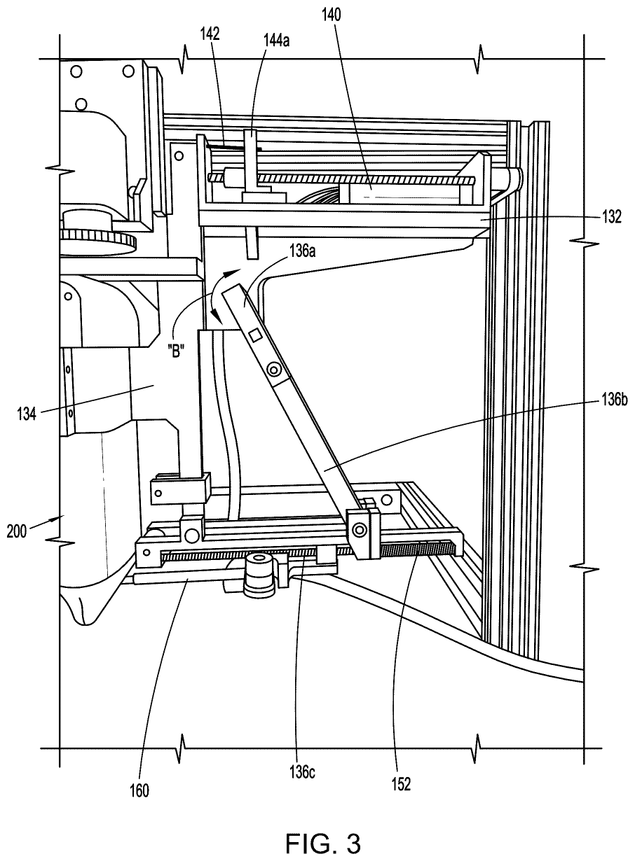

As illustrated in FIGS. 1-5, the needle manipulation assembly 100 includes a housing or frame 102, and a needle control assembly 110. The needle control assembly 110 includes a first rotation or yaw control mechanism 120 supported on the frame 102. The first rotation control mechanism 120 includes a first motor or yaw control motor 122 defining a first motor drive axis “Y1”, and a drive gear 122a connected to the first motor 122 and rotatable by the first motor 122 about the first motor drive axis “Y1”. The first rotation control mechanism 120 further includes a driven gear 122b connected a bracket 130, wherein the driven gear 122b is rotatable about a bracket pivot axis “Y2” (described below).

It is contemplated that the first motor drive axis “Y1” is oriented orthogonal to or substantially orthogonal to a horizontal plane. The first motor 122 is electrically connected to and controlled by the controller 20.

The needle control assembly 110 includes a bracket 130 pivotally connected to the frame 102 about a bracket pivot axis “Y2”. The bracket 130 is operatively connected to the first motor 122 such that the bracket 130 may be turned in response to rotations by the first motor 122, thereby changing a yaw of needle control assembly 110. It is envisioned that the bracket pivot axis “Y2” is parallel (or substantially parallel) to the first motor drive axis “Y1”.

The bracket 130 includes a first bracket arm 132, and a second bracket arm 134. The bracket 130 supports a linkage assembly 136 including a first link arm 136a pivotally connected to the first bracket arm 132 or the second bracket arm 134, a second link arm 136b pivotally connected to the first link arm 136a, and a third link arm 136c pivotally connected to each of the second link arm 136b and the second bracket arm 134. The pivot connection of the third link arm 136c with the second bracket arm 134 defines a pivot axis “PA” which is oriented orthogonal to the bracket pivot axis “Y2”, and orthogonal to the axis of translation “X1” of the needle carriage 150, as described below.

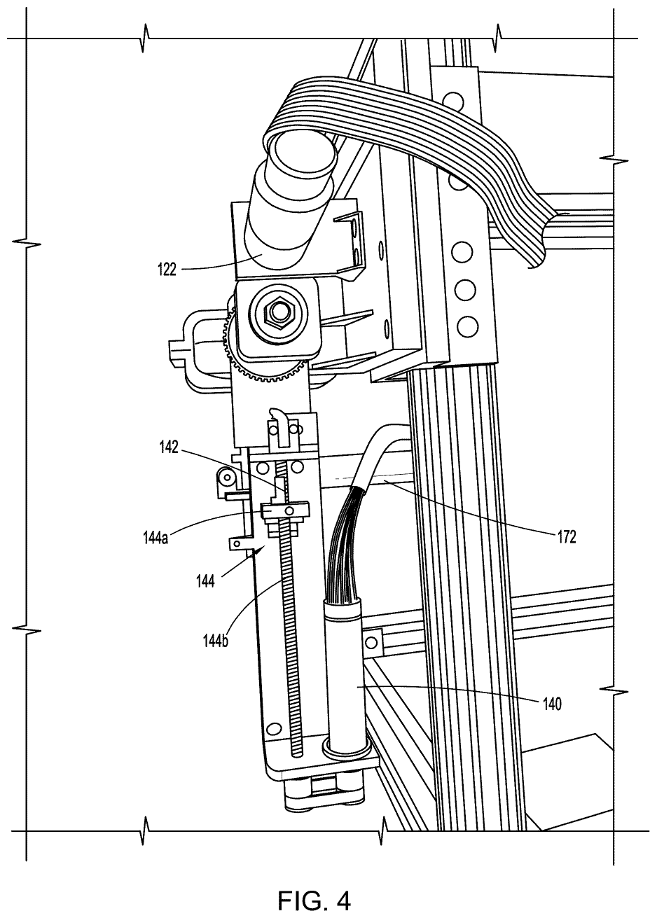

The needle control assembly 110 includes a second motor or needle drive/deployment/retraction motor 140 supported on the first bracket arm 132. The second motor 140 is connected to a first end of a cable 142 via a thread and nut arrangement 144, wherein rotation of the drive shaft of the second motor 140 causes the cable 142 to be retracted (pulled) or be let out (pushed). A second end of the cable 142 extends to and connects to a needle carriage 150 slidably supported on the third link arm 136c of the linkage assembly 136. The needle control assembly 110 includes a series of pulleys, guides or the like for guiding a path of the cable 142 from, as illustrated in FIG. 4, a nut 144a threadably connected to a threaded rod 144b of the thread and nut arrangement 144 to the needle carriage 150.

The needle carriage 150 is spring biased in a proximal direction by a biasing member 152. The biasing member 152 includes a first end fixedly secured to the third link arm 136c of the linkage assembly 136, and a second end fixedly secured to the needle carriage 150. In use, when the second motor 140 rotates in a first direction to retract the cable 142, the distal end of the cable 142 acts on the needle carriage 150 to draw the needle carriage 150 distally (as indicated by double-headed arrow “A”), and to bias the biasing member 152. Further in use, when the second motor 140 rotates in a second direction opposite the first direction (or is allowed to rotate freely), the biasing member 152 may now act on the needle carriage 150 to draw the needle carriage 150 proximally (as indicated by double-headed arrow “A”). As the needle carriage 150 is drawn proximally, the needle carriage 150 acts on the second end of the cable 142 which thereby causes the first end of the cable 142 to pull on the nut 144a of the thread and nut arrangement 144.

The needle carriage 150 is slidably supported on a rail 150a which defines an axis of translation “X1” of the needle carriage 150 (as indicated by double-headed arrow “A”).

The needle carriage 150 includes a needle clip 154 configured and adapted to releasably receive a hemodialysis needle 160. When connected to the needle clip 154 of the needle carriage 150, the needle 160 defines a longitudinal axis “X2” which may be coincident with the axis of translation “X1” of the needle carriage 150 or may be parallel (or substantially parallel) to and offset a radial distance from the axis of translation “X1” of the needle carriage 150. An orthogonal distance from the pivot axis “PA” of the third link arm 136c to the longitudinal axis “X2” of the needle 160 defines pivot radius “PR” for the needle 160. The intersection point between the pivot axis “PA” and the longitudinal axis “X2” defines a tangent point “TP”.

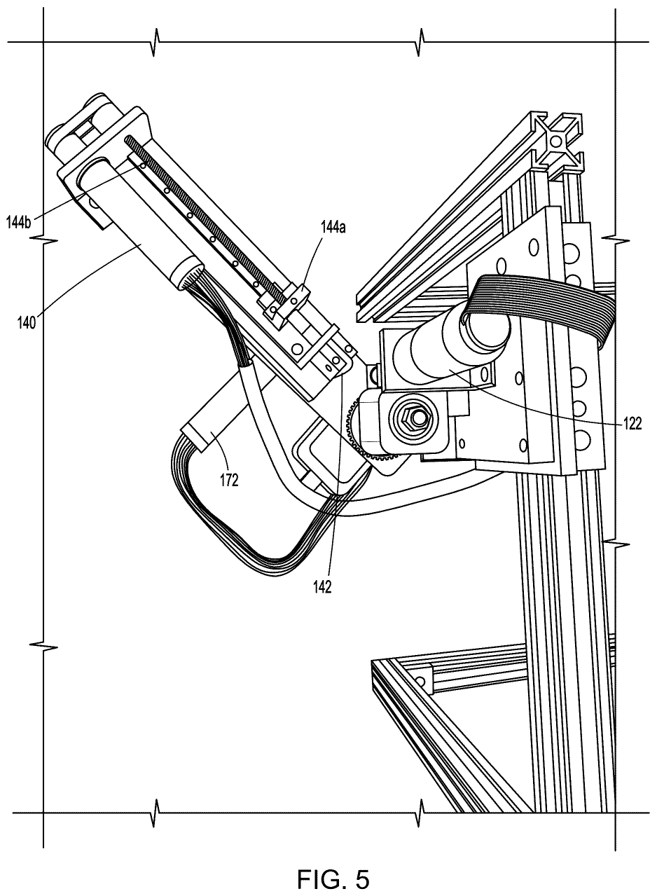

The needle control assembly 110 includes a second rotation or pitch control mechanism 170 supported on the bracket 130 and operatively connected to the first link arm 136a. The second rotation control mechanism 170 includes a third motor or pitch control motor 172 (see FIGS. 4-5) connected to the first link arm 136a. The third motor 172 is electrically connected to and controlled by the controller 20.

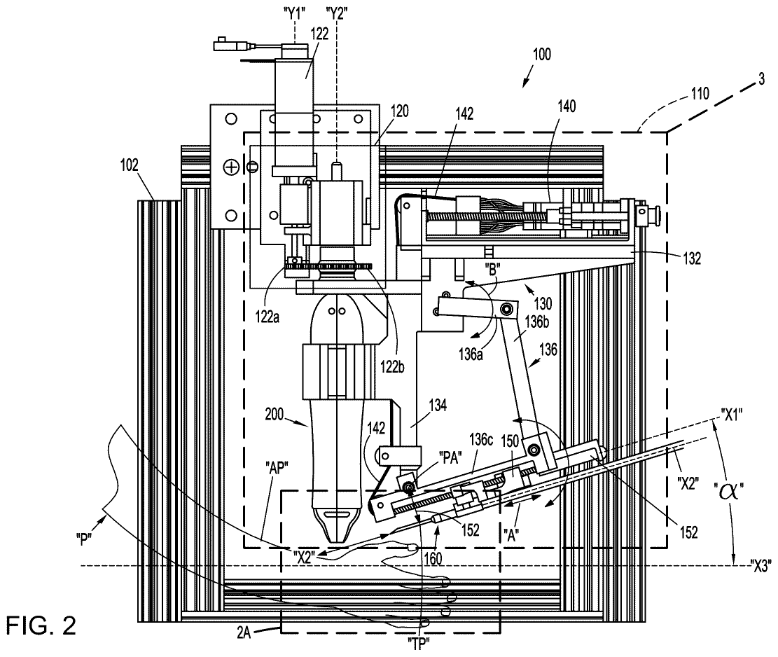

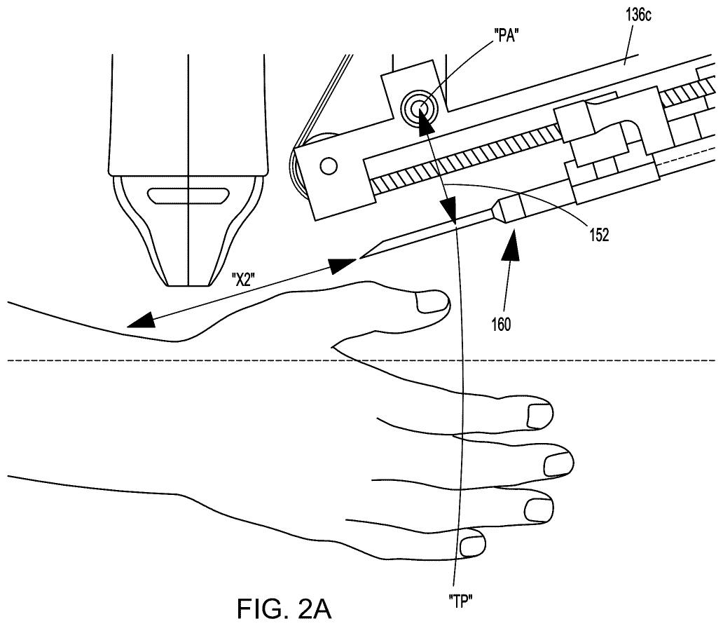

In use, when the third motor 172 rotates, in a first direction (as indicated by double-headed arrow “B” (see FIG. 2), the first link arm 136a is rotated which in turn acts on the second link arm 136b and the third link arm 136c to change an angle of inclination “α” (i.e., angle of attack or pitch) of the axis of translation “X1” of the needle carriage 150. The angle of inclination “α” is the angle between the axis of translation “X1” of the needle carriage 150, or the axis of translation “X2” of the needle 160 supported by the needle carriage 150, and either a horizontal axis, a longitudinal axis “X3” of an arm “AP” of a patient “P” positioned within the robotically-assisted hemodialysis access system 10, or a longitudinal axis of a target vein within the arm “AP” of the patient “P”.

To calculate and determine the angle of inclination “α”, with the coordinates of the tangent point “TP” known, the inverse tangent is calculated using the following formula:

“α”=tan⁻¹ (y/x)
where:
   (x, y) are the coordinates of the tangent point “TP”.

The needle manipulation assembly 100 further includes an imaging device 200 supported on bracket 130, and specifically second bracket arm 134. Imaging device 200 includes an imaging head oriented toward the needle carriage 150. More specifically, a line of sight/imaging of the imaging device 200 desirably crosses the axis of translation “X1” of the needle carriage 150. Even further, the line of sight/imaging of the imaging device 200 is aligned, or parallel (or substantially parallel), with the bracket pivot axis “Y2” of bracket 130.

The imaging device 200 may be in the form of a camera, an ultrasound imaging device, a combination of both, visible light, near-infrared light, MRI, laser ultrasonics or the like.

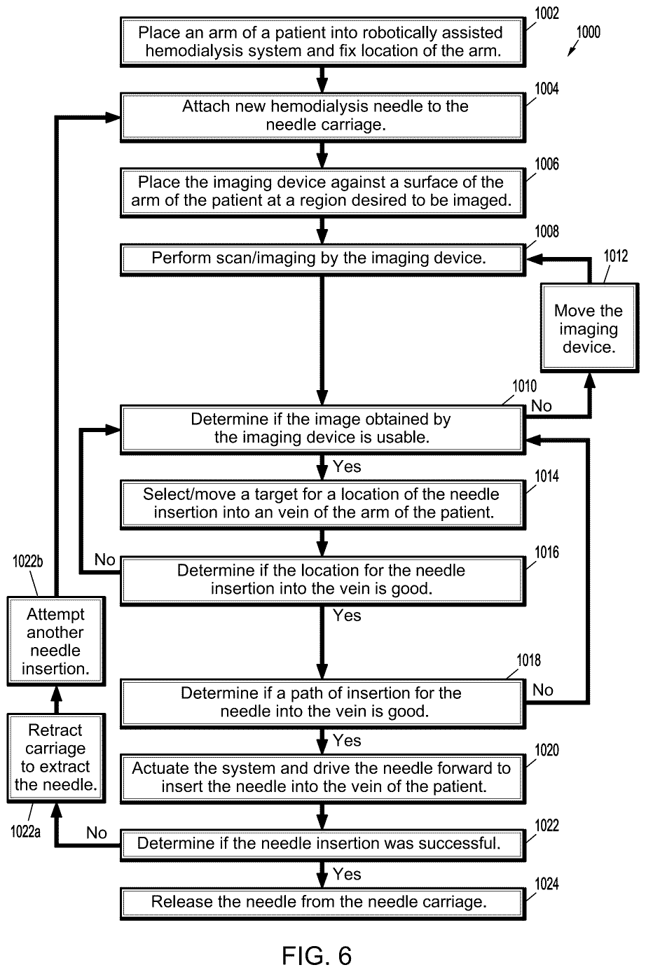

Turning now to FIG. 6, in accordance with the present disclosure, a flow chart which depicts an exemplary method 1000 for using the robotically-assisted hemodialysis access system 10 for inserting a hemodialysis needle 160 into a vein of an arm of a patient, is shown and described. The steps of method 1000 may be embodied as software instructions executable by any one or more of the controllers of the robotically-assisted hemodialysis access system 10 (e.g., controller or control processor 20, etc.).

At step 1002, an arm “AP” of a patient “P” is placed into the robotically assisted hemodialysis access system 10, and the arm “AP” is fixed to prevent or inhibit movement. At step 1004, a new hemodialysis needle 160 is attached to the needle carriage 150. At step 1006, the imaging device 200 is placed against the surface of the arm “AP” at a region of the arm “AP” that is desired to be imaged in order to obtain an image of a vein “AA” of the patient “P”. At step 1008, a scanning/imaging of the desired region of the arm “AP” to be scanned/imaged is performed.

At step 1010, a determination is made regarding the usability of the image obtained by the imaging device 200 in step 1008. Determination may be made regarding the usability of the image by confirming that the image processing software can segment the vein “AA” (or other target vessel) and identify a center point of the vein “AA”; confirming that the vein “AA” has sufficient blood flow for the hemodialysis procedure; and confirming that no stenosis is present.

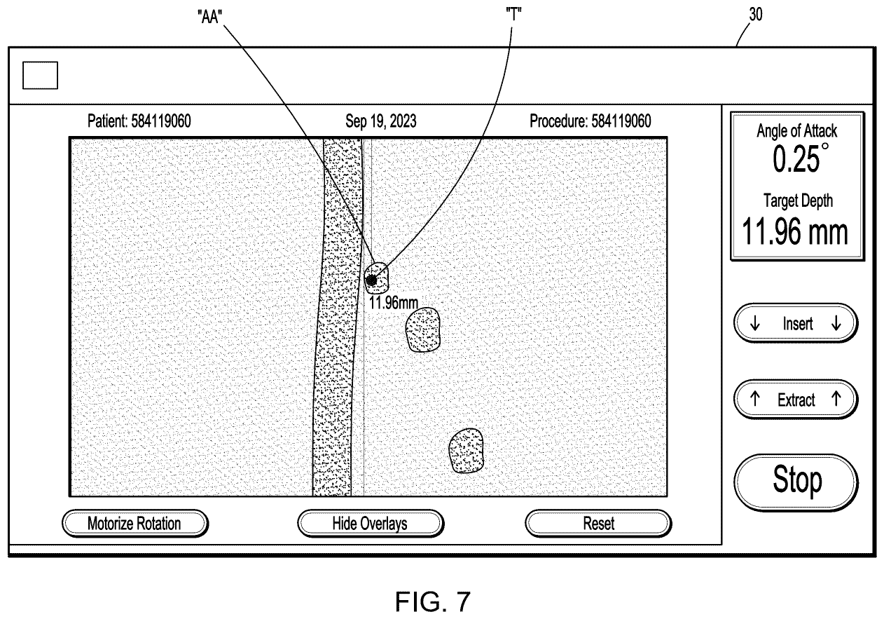

If the image obtained by the imaging device 200 in step 1008 is not usable, then, at step 1012, the imaging device 200 is moved to another region of the arm “AP” to be scanned/imaged, and step 1008 is repeated. If the image obtained by the imaging device 200 in step 1008 is usable, then, the method proceeds to step 1014, where, as depicted in FIG. 7, a target point “T” for an insertion of the needle 160 into a vein “AA” of the arm “AP” of the patient “P” is selected or moved on display 30 (see FIGS. 1 and 7).

Once the target point “T” for the needle insertion is selected at step 1014, then, at step 1016, a determination is made regarding the target point “TP” location for the insertion of the needle 160. If it is determined that the location of the target point “TP” for the needle insertion in step 1016 is not acceptable, then the method returns to step 1010 for a determination regarding the usability of the image obtained by the imaging device 200 in step 1008. If it is determined that the location of the target point “TP” for the needle insertion in step 1016 is acceptable, then the method proceeds to step 1018 wherein a determination is made regarding a path of insertion of the needle 160 into the vein “AA” of the arm “AP” of the patient “P”.

At step 1018, a determination is made as to whether a path of insertion of the needle 160 into the vein “AA” of the arm “AP” of the patient “P” is good or usable. Determination may be made regarding the usability of the path of insertion by confirming that the path of insertion will ultimately intersect the line of sight/imaging “Y2” of the imaging device; and confirming that the path of insertion will not hit an unintended blood vessel.

At step 1018, if it is determined that the path of insertion for the needle 160 into the vein “AA” of the arm “AP” of the patient “P” is not acceptable, then the method returns to step 1010 for a determination regarding the usability of the image obtained by the imaging device 200 in step 1008. If it is determined that the path of insertion for the needle 160 into the vein “AA” of the arm “AP” of the patient “P” is acceptable, then the method proceeds to step 1020.

At step 1020, the robotically-assisted hemodialysis access system 10 is activated, via display 30, a control console (not shown), or the like, to drive the needle carriage 150, and in turn, the needle forward, by a calculated insertion distance or target depth, into the vein “AA” of the arm “AP” of the patient “P”.

Also at step 1020, an insertion distance of a tip of the needle 160 is determined. With the tangent point “TP” and the angle of inclination “α” known and calculated as described above, a distance between the tangent point “TP” and the target point “T”, for where the tip of the needle 160 is to be inserted into the vein “AA” of the arm “AP” of the patient “P”, is calculated. This distance is the distance that the tip of the needle 160 is past the tangent point “TP” when the insertion motor (i.e., second motor 140 of needle control assembly 110) and the angle of inclination motor (i.e., third motor 172 of the second rotation control mechanism 170) are both at their home position. In operation, as the pitch or angle of inclination motor (i.e., third motor 172) rotates, the needle 16 is drawn forward due to the connection with the cable 142 of the needle control assembly 110. Accordingly, the change or offset in the location of the tip of the needle 160, as a function of the angle of inclination “α”, is obtained from a look-up table, and this change or offset is applied to the calculated insertion distance or target depth (i.e., added to or subtracted from the calculated insertion distance).

Following insertion of the needle 150 into the vein “AA” of the arm “AP” of the patient “P”, at step 1022 a determination is made whether the insertion of the needle 150 was successful. If it is determined that the insertion of the needle 150 into the vein “AA” of the arm “AP” of the patient “P” was not successful, then the method proceeds to step 1022a, wherein the needle carriage 150 is retracted, and then in step 1022b, if insertion of another (new) needle 160 is desired, the method returns to step 1004.

If it is determined that the insertion of the needle 150 into the vein “AA” of the arm “AP” of the patient “P” was successful, then the method proceeds to step 1024 and the needle 160 is released from the needle carriage 150.

The determinations at steps 1010, 1016, 1018 and 122 may be conducted by a technician, by a machine learning algorithm, or a combination of both. The machine learning algorithm of the present disclosure may be used to monitor, observe and, when desired/necessary, react to or provide an alert related to fistula cannulation and insertion, robotic control memory, optimization of second cannulation, detecting the vein the ultrasound images, detecting when the needle tip has entered the vasculature and preventing puncture of the back-wall of the vasculature (i.e., vein or artery), interactions with the patient to provide emotional support during the cannulation, or the like. In regard to the machine learning model used for detecting the vein in the ultrasound images, the machine learning algorithm may be trained using B-mode and color doppler images.

Further aspects and embodiments of the present disclosure are set out in the below numbered clauses:
1. A robotically-assisted hemodialysis access system, comprising:
a controller;
a display connected to the controller; and
a needle manipulation assembly connected to the controller, the needle manipulation assembly including:
   a frame; and
   a needle control assembly having:
      a first rotation control mechanism for rotating the needle control assembly about a first axis, wherein the first axis is oriented substantially orthogonal to a horizontal plane, wherein the first rotation control mechanism includes a first motor defining a first motor drive axis;
      a bracket pivotally connected to the frame about a bracket pivot axis, wherein the bracket pivot axis is substantially parallel to the first motor drive axis, wherein an orientation of the bracket is controlled by the first motor, wherein the bracket pivot axis is axially aligned with the first axis, and wherein the bracket includes:
         a first bracket arm;
         a second bracket arm extending transverse to the first bracket arm; and
         a linkage assembly including a first link arm pivotally connected to the first bracket arm, a second link arm pivotally connected to the first link arm, and a third link arm pivotally connected to each of the second link arm and the second bracket arm;
      a needle carriage slidably connected to the third link arm, wherein the needle carriage translates relative to the third link arm, wherein a path of translation of the needle carriage defines an axis of translation;
      a second motor supported on the bracket, wherein the second motor is operatively connected to a first end of a cable, wherein a second end of the cable is operatively connected to the needle carriage, and wherein a position of the needle carriage is controlled by the second motor; and
      a second rotation control mechanism supported on the bracket and operatively connected to the first link arm, the second rotation control mechanism including a third motor connected to the first link arm, wherein an angle of inclination of the axis of translation of the needle carriage is controlled by the third motor; and
an imaging device supported on the bracket, wherein the imaging device includes an imaging head defining a line of sight that crosses the axis of translation of the needle carriage and that is substantially parallel with the bracket pivot axis.
2. The robotically-assisted hemodialysis access system according to clause 1, wherein the first rotation control mechanism includes:
a drive gear connected to the first motor; and
a driven gear supported on the bracket, the driven gear defining a driven gear rotation axis,
wherein the driven gear rotation axis is rotatably aligned with the bracket pivot axis.
3. The robotically-assisted hemodialysis access system according to clause 1 or 2, wherein rotation of the first motor results in rotation of the driven gear and rotation of the bracket about the bracket pivot axis to adjust a yaw of the bracket.
4. The robotically-assisted hemodialysis access system according to any of the preceding clauses, wherein the pivot connection of the third link arm with the second bracket arm defines a needle carriage pivot axis, wherein the needle carriage pivot axis is oriented orthogonal to both the bracket pivot axis and the axis of translation of the needle carriage.
5. The robotically-assisted hemodialysis access system according to any of the preceding clauses, wherein the second motor is connected to the first end of the cable via a thread and nut arrangement, wherein rotation of the drive shaft of the second motor causes the cable to be actuated.
6. The robotically-assisted hemodialysis access system according to any of the preceding clauses, wherein the needle control assembly includes a series of pulleys for guiding a path of the cable from a nut of the thread and nut arrangement to the needle carriage.
7. The robotically-assisted hemodialysis access system according to any of the preceding clauses, wherein the needle carriage is spring-biased in a proximal direction by a biasing member.
8. The robotically-assisted hemodialysis access system according to any of the preceding clauses, wherein, when the second motor rotates in a first direction and retracts the cable, the distal end of the cable acts on the needle carriage to draw the needle carriage distally, and to bias the biasing member.
9. The robotically-assisted hemodialysis access system according to any of the preceding clauses, wherein the needle carriage includes a needle clip for releasably receiving a needle.
10. The robotically-assisted hemodialysis access system according to any of the preceding clauses, wherein, when the third motor rotates, the first link arm is rotated which in turn acts on the second link arm and the third link arm to change the angle of inclination of the axis of translation of the needle carriage.
11. The robotically-assisted hemodialysis access system according to any of the preceding clauses, wherein the imaging device is an ultrasound imaging device.
12. The robotically-assisted hemodialysis access system according to any of the preceding clauses, wherein the first motor, the second motor, and the third motor are electrically connected to and controlled by the controller.
13. A robotically-assisted hemodialysis access system, comprising:
a controller;
a display connected to the controller; and
a needle manipulation assembly connected to the controller, the needle manipulation assembly including:
   a frame; and
   a needle control assembly having:
      a needle carriage slidably connected to the frame, wherein a path of translation of the needle carriage defines an axis of translation;
      a needle drive motor operatively connected to a first end of a cable, wherein a second end of the cable is operatively connected to the needle carriage, and wherein a position of the needle carriage is controlled by the needle drive motor; and
      a pitch control mechanism including a pitch motor connected to operatively connected to the needle carriage, wherein an angle of inclination of the axis of translation of the needle carriage is controlled by the pitch motor; and
an imaging device supported on the bracket, wherein the imaging device includes an imaging head defining a line of sight that crosses the axis of translation of the needle carriage.
14. The robotically-assisted hemodialysis access system according to clause 13, wherein the needle control assembly includes a yaw control mechanism for rotating the needle control assembly about a first axis, wherein the first axis is oriented substantially orthogonal to a horizontal plane, and wherein the yaw control mechanism includes a yaw control motor defining a yaw motor drive axis.
15. The robotically-assisted hemodialysis access system according to clause 13 or 14, wherein:
the needle control assembly comprises:
   a bracket connected to the frame, wherein the bracket includes:
      a first bracket arm;
      a second bracket arm extending transverse to the first bracket arm; and
      a linkage assembly including a first link arm pivotally connected to the first bracket arm, a second link arm pivotally connected to the first link arm, and a third link arm pivotally connected to each of the second link arm and the second bracket arm; and
the bracket is pivotally connected to the frame about a bracket pivot axis, wherein the bracket pivot axis is substantially parallel to the yaw motor drive axis, wherein a yaw of the bracket is controlled by the yaw control motor, and wherein the bracket pivot axis is axially aligned with the first axis.
16. The robotically-assisted hemodialysis access system according to any of clauses 13-15, wherein the pivot connection of the third link arm with the second bracket arm defines a needle carriage pivot axis, wherein the needle carriage pivot axis is oriented orthogonal to both the bracket pivot axis and the axis of translation of the needle carriage.
17. The robotically-assisted hemodialysis access system according to any of clauses 13-16, wherein the imaging device is an ultrasound imaging device.
18. A method of inserting a hemodialysis needle into a vein of an arm of a patient, using a robotically-assisted hemodialysis access system, comprises:
placing an imaging device at a desired region of the arm of the patient;
imaging the desired region of the arm of the patient with the imaging device to obtain an image;
determining a usability of the image obtained by the imaging device;
selecting a target point for an insertion of the needle into the vein of the arm of the patient;
determining an acceptability of a location of the target point for the insertion of the needle into the vein of the arm of the patient;
determining a path of insertion of the needle into the vein of the arm of the patient including an angle of inclination for the path of insertion;
driving the needle distally, by a calculated insertion distance as a function of the angle of inclination, into the vein of the arm of the patient; and
determining an acceptability of the insertion of the needle into the vein of the arm of the patient.
19. The method according to clause 18, wherein when the determining of the usability of the image obtained by the imaging device is determined to be unacceptable, the imaging device is placed at another desired position, another imaging is performed with the imaging device.
20. The method according to clause 18 or 19, wherein when:
the determining of the acceptability of the location of the target point for the insertion of the needle into the vein of the arm of the patient is determined to be unacceptable; or
the determining of the path of insertion of the needle into the vein of the arm of the patient is determined to be unacceptable;
the usability of the image obtained by the imaging device is redetermined.
21. The method according to any of clauses 18-20, wherein when the acceptability of the insertion of the needle into the vein of the arm of the patient is determined to be unacceptable, then the method includes:
retracting the needle;
determining if another needle insertion is desired; and
attaching a new needle to the robotically assisted hemodialysis system prior to performing the another needle insertion.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A robotically-assisted hemodialysis access system, comprising:
a controller;
a display connected to the controller; and
a needle manipulation assembly connected to the controller, the needle manipulation assembly including:
a frame; and
a needle control assembly having:
a first rotation control mechanism for rotating the needle control assembly about a first axis, wherein the first axis is oriented substantially orthogonal to a horizontal plane, wherein the first rotation control mechanism includes a first motor defining a first motor drive axis;
a bracket pivotally connected to the frame about a bracket pivot axis, wherein the bracket pivot axis is substantially parallel to the first motor drive axis, wherein an orientation of the bracket is controlled by the first motor, wherein the bracket pivot axis is axially aligned with the first axis, and wherein the bracket includes:
a first bracket arm;
a second bracket arm extending transverse to the first bracket arm; and
a linkage assembly including a first link arm pivotally connected to the first bracket arm, a second link arm pivotally connected to the first link arm, and a third link arm pivotally connected to each of the second link arm and the second bracket arm;
a needle carriage slidably connected to the third link arm, wherein the needle carriage translates relative to the third link arm, wherein a path of translation of the needle carriage defines an axis of translation;
a second motor supported on the bracket, wherein the second motor is operatively connected to a first end of a cable, wherein a second end of the cable is operatively connected to the needle carriage, and wherein a position of the needle carriage is controlled by the second motor; and
a second rotation control mechanism supported on the bracket and operatively connected to the first link arm, the second rotation control mechanism including a third motor connected to the first link arm, wherein an angle of inclination of the axis of translation of the needle carriage is controlled by the third motor; and
an imaging device supported on the bracket, wherein the imaging device includes an imaging head defining a line of sight that crosses the axis of translation of the needle carriage and that is substantially parallel with the bracket pivot axis.

2. The robotically-assisted hemodialysis access system according to claim 1, wherein the first rotation control mechanism includes:
a drive gear connected to the first motor; and
a driven gear supported on the bracket, the driven gear defining a driven gear rotation axis,
wherein the driven gear rotation axis is rotatably aligned with the bracket pivot axis.

3. The robotically-assisted hemodialysis access system according to claim 2, wherein rotation of the first motor results in rotation of the driven gear and rotation of the bracket about the bracket pivot axis to adjust a yaw of the bracket.

4. The robotically-assisted hemodialysis access system according to claim 1, wherein the pivot connection of the third link arm with the second bracket arm defines a needle carriage pivot axis, wherein the needle carriage pivot axis is oriented orthogonal to both the bracket pivot axis and the axis of translation of the needle carriage.

5. The robotically-assisted hemodialysis access system according to claim 1, wherein the second motor is connected to the first end of the cable via a thread and nut arrangement, wherein rotation of the drive shaft of the second motor causes the cable to be actuated.

6. The robotically-assisted hemodialysis access system according to claim 5, wherein the needle control assembly includes a series of pulleys for guiding a path of the cable from a nut of the thread and nut arrangement to the needle carriage.

7. The robotically-assisted hemodialysis access system according to claim 1, wherein the needle carriage is spring-biased in a proximal direction by a biasing member.

8. The robotically-assisted hemodialysis access system according to claim 7, wherein, when the second motor rotates in a first direction and retracts the cable, the distal end of the cable acts on the needle carriage to draw the needle carriage distally, and to bias the biasing member.

9. The robotically-assisted hemodialysis access system according to claim 1, wherein the needle carriage includes a needle clip for releasably receiving a needle.

10. The robotically-assisted hemodialysis access system according to claim 1, wherein, when the third motor rotates, the first link arm is rotated which in turn acts on the second link arm and the third link arm to change the angle of inclination of the axis of translation of the needle carriage.

11. The robotically-assisted hemodialysis access system according to claim 1, wherein the imaging device is an ultrasound imaging device.

12. The robotically-assisted hemodialysis access system according to claim 1, wherein the first motor, the second motor, and the third motor are electrically connected to and controlled by the controller.

13. A robotically-assisted hemodialysis access system, comprising:
a controller;
a display connected to the controller; and
a needle manipulation assembly connected to the controller, the needle manipulation assembly including:
a frame; and
a needle control assembly having:
a needle carriage slidably connected to the frame, wherein a path of translation of the needle carriage defines an axis of translation;
a needle drive motor operatively connected to a first end of a cable, wherein a second end of the cable is operatively connected to the needle carriage, and wherein a position of the needle carriage is controlled by the needle drive motor; and
a pitch control mechanism including a pitch motor connected to operatively connected to the needle carriage, wherein an angle of inclination of the axis of translation of the needle carriage is controlled by the pitch motor; and
an imaging device supported on the bracket, wherein the imaging device includes an imaging head defining a line of sight that crosses the axis of translation of the needle carriage.

14. The robotically-assisted hemodialysis access system according to claim 13, wherein the needle control assembly includes a yaw control mechanism for rotating the needle control assembly about a first axis, wherein the first axis is oriented substantially orthogonal to a horizontal plane, and wherein the yaw control mechanism includes a yaw control motor defining a yaw motor drive axis.

15. The robotically-assisted hemodialysis access system according to claim 14, wherein:
the needle control assembly comprises:
a bracket connected to the frame, wherein the bracket includes:
a first bracket arm;
a second bracket arm extending transverse to the first bracket arm; and
a linkage assembly including a first link arm pivotally connected to the first bracket arm, a second link arm pivotally connected to the first link arm, and a third link arm pivotally connected to each of the second link arm and the second bracket arm; and
the bracket is pivotally connected to the frame about a bracket pivot axis, wherein the bracket pivot axis is substantially parallel to the yaw motor drive axis, wherein a yaw of the bracket is controlled by the yaw control motor, and wherein the bracket pivot axis is axially aligned with the first axis.

16. The robotically-assisted hemodialysis access system according to claim 15, wherein the pivot connection of the third link arm with the second bracket arm defines a needle carriage pivot axis, wherein the needle carriage pivot axis is oriented orthogonal to both the bracket pivot axis and the axis of translation of the needle carriage.

17. The robotically-assisted hemodialysis access system according to claim 13, wherein the imaging device is an ultrasound imaging device.

18. A method of inserting a hemodialysis needle into a vein of an arm of a patient using a robotically-assisted hemodialysis access system, the method comprising:
placing an imaging device at a desired region of the arm of the patient;
imaging the desired region of the arm of the patient with the imaging device to obtain an image;
determining a usability of the image obtained by the imaging device;
selecting a target point for an insertion of the needle into the vein of the arm of the patient;
determining an acceptability of a location of the target point for the insertion of the needle into the vein of the arm of the patient;
determining a path of insertion of the needle into the vein of the arm of the patient including an angle of inclination for the path of insertion;
driving the needle distally, by a calculated insertion distance as a function of the angle of inclination, into the vein of the arm of the patient; and
determining an acceptability of the insertion of the needle into the vein of the arm of the patient.

19. The method according to claim 18, wherein when the determining of the usability of the image obtained by the imaging device is determined to be unacceptable, the imaging device is placed at another desired position, another imaging is performed with the imaging device.

20. The method according to claim 18, wherein when:
the determining of the acceptability of the location of the target point for the insertion of the needle into the vein of the arm of the patient is determined to be unacceptable; or
the determining of the path of insertion of the needle into the vein of the arm of the patient is determined to be unacceptable;
the usability of the image obtained by the imaging device is redetermined.
